(19) United States
(12) Patent Application Publication
(10) Pub. No.: US 2010/0260716 A1
(43) Pub. Date: Oct. 14, 2010

(54) COMPOUNDS FOR TREATING DEMYELINATION CONDITIONS

(75) Inventors: Thomas Stöhr, Monheim (DE); David Rudd, Raleigh, NC (US)

Correspondence Address: HARNESS, DICKEY, & PIERCE, P.L.C, 7700 Bonhomme, Suite 400, ST. LOUIS, MO, 63105, US

(73) Assignee: UCB PHARMA GMBH, Monheim (DE)

(21) Appl. No.: 12/682,852

(22) PCT Filed: Oct. 23, 2008
(86) PCT No.: PCT/EP08/08969

§ 371 Date: Jun. 8, 2010

Related U.S. Application Data
(60) Provisional application No. 60/981,842, filed on Oct. 23, 2007.

(51) Int. Cl. A61K 38/21; A61K 31/165; A61K 38/07; A61K 31/568; A61K 31/4355; A61K 39/395; A61K 31/7076; A61K 31/47; A61K 31/198; A61P 21/00

(52) U.S. Cl. 424/85.6; 514/626; 514/17.7; 514/171; 514/291; 424/141.1; 514/46; 514/312; 514/563

(57) ABSTRACT

Therapeutic methods, therapeutic combinations and pharmaceutical compositions provided herein are useful for inhibiting demyelination, for delaying the clinical onset of a demylination condition, for inhibiting progression and/or reducing frequency of relapse of a demylination condition, and/or enhancing physical ability of a human subject having a demylination condition. Lacosamide is one of the active compounds.

### FIELD OF THE INVENTION

The present invention relates to therapeutic methods, therapeutic combinations and pharmaceutical compositions useful for treating demyelination conditions.

### BACKGROUND OF THE INVENTION

Demyelination is a degenerative process causing erosion of the myelin sheath that normally protects nerve fibers. Demyelination exposes these fibers and appears to cause problems in nerve impulse conduction that may affect many physical systems. Demyelination is seen in a number of diseases, for example, in multiple sclerosis.

Multiple sclerosis is a debilitating, inflammatory, neurological demyelinating disease that affects the central nervous system (CNS). Multiple sclerosis causes gradual demyelination which leaves scar tissue called sclerosis throughout the brain and spinal cord. These damaged areas are also known as plaques or lesions. Sometimes the axon of the nerve fiber itself is damaged or broken. While the exact etiology of multiple sclerosis is unknown, multiple sclerosis is currently believed to involve an autoimmune response.

United States Patent Application Publication No. 2002/0119944 of Aguera et al. relates to methods for the prevention or treatment of myelin disorders by modulating a Ulip/CRMP activity. Examples of myelin disorders mentioned therein include multiple sclerosis, HTLV-1 associated myelopathy and leucodystrophies.

Certain peptides are known to exhibit CNS activity and are useful in the treatment of epilepsy and other CNS disorders. Such peptides are described, for example, in U.S. Pat. No. 5,378,729 to Kohn & Watson.

Related peptides are disclosed in U.S. Pat. No. 5,773,475 to Kohn as useful for treating CNS disorders.

International Patent Publication No. WO 2005/120476 relates to use of such peptide compounds for treatment of motoneuron disorders, particularly amyotrophic lateral sclerosis, and peripheral neuropathies such as Guillain-Barré syndrome and Charcot-Marie-Tooth syndrome.

International Patent Publication No. WO 2005/110390 relates to use of such peptide compounds for treatment of primary and/or secondary dyskinesia. It is stated therein that “[s]econdary dyskinesias may be observed in various diseases either as a secondary symptom (head injury, multiple sclerosis) or as a consequence of drug treatments.”

International Patent Publication No. WO 2005/053667 relates to use of such peptide compounds for treatment of central neuropathic pain. It is stated therein that a variety of CNS diseases such as multiple sclerosis, myelitis, syphilis, ischemia, hemorrhages or arteriovenous malformations may be associated with central neuropathic pain.

International Patent Publication No. WO 2006/079547 relates to use of such peptide compounds for treatment of a disease treated with antipsychotics, in particular psychosis and schizophrenia, in an add-on therapy to at least one antipsychotic. It is stated therein that some non-psychiatric conditions, which may include brain tumor, dementia with Lewy bodies, hypoglycemia, intoxication, multiple sclerosis, systemic lupus erythematosus and/or sarcoidosis, are linked to psychosis.

Approximately 400,000 Americans acknowledge having multiple sclerosis, and every week about 200 people are diagnosed. Multiple sclerosis may affect 2.5 million individuals worldwide. See http://www.nationalmssociety.org/site/PageServer?pagename=HOM_ABOUT_who_gets_ms.

Therefore, a need remains for improved therapies for persons having a demyelination condition, particularly multiple sclerosis. In particular, a need remains for such therapies that can address the demyelination condition itself, not limited to alleviation of secondary effects or symptoms of the condition such as dyskinesia, neuropathic pain or psychosis.

### SUMMARY OF THE INVENTION

There is now provided a method for inhibiting demyelination in a human subject having a demyelination condition, the method comprising administering to the subject a compound of Formula (I)

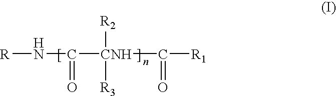

wherein
R is hydrogen, lower alkyl, lower alkenyl, lower alkynyl, aryl, aryl lower alkyl, heterocyclic, heterocyclic lower alkyl, lower alkyl heterocyclic, lower cycloalkyl or lower cycloalkyl lower alkyl, and R is unsubstituted or is substituted with at least one electron withdrawing group and/or at least one electron donating group;
R₁ is hydrogen or lower alkyl, lower alkenyl, lower alkynyl, aryl lower alkyl, aryl, heterocyclic lower alkyl, lower alkyl heterocyclic, heterocyclic, lower cycloalkyl, lower cycloalkyl lower alkyl, each unsubstituted or substituted with at least one electron donating group and/or at least one electron withdrawing group;
R₂ and R₃ are independently hydrogen, lower alkyl, lower alkenyl, lower alkynyl, aryl lower alkyl, aryl, halo, heterocyclic, heterocyclic lower alkyl, lower alkyl heterocyclic, lower cycloalkyl, lower cycloalkyl lower alkyl, or Z—Y, wherein R₂ and R₃ may be unsubstituted or substituted with at least one electron withdrawing group and/or at least one electron donating group; and wherein heterocyclic in R₂ and R₃ is furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indtolyl, purinyl, indolinyl, pyrazolindinyl, imidazolinyl, imidazolindinyl, pyrrolidinyl, furazanyl, N-methylindolyl, methylfuryl, pyridazinyl, pyrimidinyl, pyrazinyl, pyridyl, epoxy, aziridino, oxetanyl, azetidinyl or, when N is present in the heterocyclic, an N-oxide thereof;
Z is O, S, S(O)ₐ, NR₄, NR₆′, PR₄ or a chemical bond;
Y is hydrogen, lower alkyl, aryl, aryl lower alkyl, lower alkenyl, lower alkynyl, halo, heterocyclic, heterocyclic lower alkyl, lower alkyl heterocyclic and Y may be unsubstituted or substituted with at least one electron donating group and/or at least one electron withdrawing group, wherein heterocyclic has the same meaning as in R₂ or R₃ and, provided that when Y is halo, Z is a chemical bond, or
Z—Y taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR₄NR₅R₇, N+R₅R₆R₇,

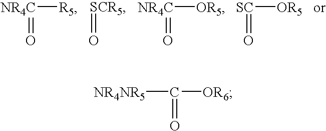

R₆′ is hydrogen, lower alkyl, lower alkenyl, or lower allynyl which may be unsubstituted or substituted with at least one electron withdrawing group and/or at least one electron donating group;
R₄, R₅ and R₆ are independently hydrogen, lower alkyl, aryl, aryl lower alkyl, lower alkenyl, or lower alkynyl, wherein R₄, R₅ and R₆ may independently be unsubstituted or substituted with at least one electron withdrawing group and/or at least one electron donating group;
R₇ is R₆ or COOR₈ or COR₈, which R₇ may be unsubstituted or substituted with at least one electron withdrawing group and/or at least one electron donating group;
R₈ is hydrogen or lower alkyl, or aryl lower alkyl, and the aryl or alkyl group may be unsubstituted or substituted with at least one electron withdrawing group and/or at least one electron donating group;
n is 1-4; and
a is 1-3;
   or a pharmaceutically acceptable salt thereof; at a dose and frequency effective to inhibit demyelination when continued for a period of at least about 3 months.

There is further provided a method for delaying clinical onset of a demyelination condition in a human subject. The method comprises administering to the subject a compound of Formula (I) or a pharmaceutically acceptable salt thereof in a therapeutically effective amount.

There is still further provided a method for inhibiting progression and/or reducing frequency of relapse of a demyelination condition in a human subject. The method comprises administering to the subject a compound of Formula (I) or a pharmaceutically acceptable salt thereof in a therapeutically effective amount for a period of at least about 3 months.

There is still further provided a method for enhancing physical ability of a human subject having a demyelination condition. The method comprises administering to the subject a compound of Formula (I) or a pharmaceutically acceptable salt thereof in a therapeutically effective amount for a period of at least about 3 months.

There is still further provided a therapeutic combination (for example in the form of a pharmaceutical composition) comprising
(a) a compound of Formula (I) or a pharmaceutically acceptable salt thereof, and
(b) at least one further active agent for treatment of multiple sclerosis or a variant thereof.

An illustrative compound of Formula (I) is lacosamide, (R)-2-acetamido-N-benzyl-3-methoxypropionamide (also called SPM 927 or harkoseride).

Other embodiments, including particular aspects of the embodiments summarized above, will be evident from the detailed description that follows.

### DETAILED DESCRIPTION

Therapeutic methods, therapeutic combinations and pharmaceutical compositions provided herein are useful for inhibiting demyelination, for delaying clinical onset of a demyelination condition, for inhibiting progression and/or reducing frequency of relapse of a demyelination condition, and/or enhancing physical ability of a human subject having a demyelination condition.

The terms “inhibit”, “inhibiting” and “inhibition” herein include to reverse, arrest, slow, retard or stabilize demyelination, progression of a demyelination condition, or an effect of such progression. For example, in some embodiments inhibition is only partial, such as a slowing or retarding of progression of a demyelination condition. In other embodiments, inhibition is more complete, such as an arrest or even reversal of such progression.

The term “demyelination condition” herein refers to a disease, disorder or syndrome in which at least one demyelinating event has occurred. A “demyelinating event” can be a directly observed demyelination lesion or a lesion inferred from a sign or symptom including, but not limited to, optic neuritis, numbness or tingling in a limb, difficulty with speech, loss of balance or coordination, or other motor or sensory problems. In certain embodiments, the demyelination condition is associated with an autoimmune response. Examples of demyelination conditions include, but are not limited to, multiple sclerosis and variants thereof, transverse myelitis, Guillain-Barré syndrome and progressive multifocal leukoencephalopathy. Variants of multiple sclerosis include, but are not limited to, optic-spinal multiple sclerosis, neuromyelitis optica, acute disseminated encephalomyelitis, Balo concentric sclerosis, Schilder disease and Marburg multiple sclerosis. In a particular embodiment, the demyelination condition comprises multiple sclerosis or a variant thereof. In a further particular embodiment, the demyelination condition is selected from multiple sclerosis and variants thereof, transverse myelitis, and progressive multifocal leukoencephalopathy.

The demyelination condition treated by a method of the invention may be, but is not necessarily, clinically diagnosed. For example, in one embodiment of the method a compound of Formula (I) or salt thereof is administered after the subject is clinically diagnosed with a demyelination condition such as multiple sclerosis. In an alternative embodiment, the subject has experienced at least one demyelinating event but a demyelination condition has not yet been clinically diagnosed. In this embodiment of the method, a compound of Formula (I) or salt thereof is administered before the subject is clinically diagnosed with a demyelination condition such as multiple sclerosis.

In one embodiment, when the subject has experienced at least one demyelinating event but is not yet clinically diagnosed with a demyelination condition, administering a compound of Formula (I) or salt thereof may delay clinical onset of the demyelination condition. In a particular embodiment, clinical onset of multiple sclerosis is delayed. The term “clinical onset” refers to a demyelinating event that confirms diagnosis of the demyelination condition. For example, in the case of multiple sclerosis, clinical onset is at least a second demyelinating event which occurs at least 30 days after a first demyelinating event.

Multiple sclerosis (MS) and other demyelination conditions can result in both neurological (including psychological) and physical effects. Physical effects may induce or result in disability. Initial attacks, i.e., acute outward manifestations of the condition, are often transient, mild or substantially asymptomatic, and are often self-limited. Later attacks, or “relapse”, are often more severe and may be punctuated by periods of remission. Severity and frequency of attacks can be used to classify MS and/or variants thereof into several subtypes:
(a) relapse-remitting MS, characterized by unpredictable attacks which may or may not leave permanent neurological deficit and/or disability, followed by periods of remission;
(b) primary progressive MS, characterized by a steady decline without attacks;
(c) secondary progressive MS, characterized by an initial relapse-remitting period followed by decline without periods of remission; and
(d) progressive relapsing MS, characterized by a steady decline since onset with superimposed attacks.

In one embodiment a compound of Formula (I) or a salt thereof is administered to inhibit demyelination in a subject having either relapse-remitting, primary progressive, secondary progressive or progressive relapsing MS or variant thereof.

Further, there are four identified lesion or scar patterns of MS and variants thereof which may or may not correlate with differences in disease type and prognosis. The four identified patterns are associated with various immune system responses and are as follows.
(a) Pattern I: The scar presents T-cells and macrophages around blood vessels, with preservation of oligodendrocytes, but no signs of complement system activation.
(b) Pattern II: The scar presents T-cells and macrophages around blood vessels, with preservation of oligodendrocytes as in Pattern I, but also signs of complement system activation can be found.
(c) Pattern III: The scars are diffuse with inflammation, distal oligodendrogliopathy and microglial activation. There is also loss of myelin-associated glycoprotein (MAG). The scars do not surround the blood vessels, and in fact a rim of preserved myelin appears around the vessels. There is evidence of partial remyelinization and oligodendrocyte apoptosis. Cases of Balo concentric sclerosis may have this pattern.
(d) Pattern IV: The scar presents sharp borders and oligodendrocyte degeneration, with a rim of normal-appearing white matter. There is a lack of oligodendrocytes in the center of the scar. There is no complement activation or MAG loss. Many cases of primary progressive MS have this pattern.

In another embodiment a method for inhibiting progression and/or reducing frequency of relapse of a demyelination condition is provided. For example, in one embodiment disability progression of MS or a variant thereof may be inhibited. Disability progression refers to physical disability which may or may not be accompanied by neurological symptoms. Examples of such physical disability include, but are not limited to, muscle weakness, abnormal muscle spasms, difficulty in moving such as ambulatory impairment, difficulties with coordination or balance, fatigue, and bladder or bowel difficulties. Disability progression may be quantified on a scale such as the Kurtzke expanded disability status scale (EDSS). The EDSS quantifies disability in eight functional systems (FS's) and allows neurologists to assign a functional system score (FSS) in each. The FS's are:
(1) pyramidal;
(2) cerebellar;
(3) brainstem;
(4) sensory;
(5) bowel and bladder;
(6) visual;
(7) cerebral; and
(8) other.

Results on the EDSS are recorded as steps 1 to 10. EDSS steps 1.0 to 4.5 refer to people with multiple sclerosis who are fully ambulatory. EDSS steps 5.0 to 9.5 are defined by impairment of ambulation. The clinical meaning of each possible result (step) is as follows.
0.0: Normal neurological exam.
1.0: No disability, minimal signs on 1 FS.
1.5: No disability, minimal signs on 2 of 7 FS.
2.0: Minimal disability in 1 of 7 FS.
2.5: Minimal disability in 2 FS.
3.0: Moderate disability in 1 FS; or mild disability in 3-4 FS, though fully ambulatory.
3.5: Fully ambulatory but with moderate disability in 1 FS and mild disability in 1 or 2 FS; or moderate disability in 2 FS; or mild disability in 5 FS.
4.0: Fully ambulatory without aid, up and about 12 hours a day despite relatively severe disability. Able to walk 500 m without aid.
4.5: Fully ambulatory without aid, up and about much of day, able to work a full day, may otherwise have some limitations of full activity or require minimal assistance. Relatively severe disability. Able to walk 300 m without aid.
5.0: Ambulatory without aid for about 200 m. Disability impairs full daily activities.
5.5: Ambulatory for 100 m, disability precludes full daily activities.
6.0: Intermittent or unilateral constant assistance (cane, crutch or brace) required to walk 100 m with or without resting.
6.5: Constant bilateral support (cane, crutch or braces) required to walk 20 m without resting.
7.0: Unable to walk beyond 5 m even with aid, essentially restricted to wheelchair, wheels self, transfers alone; active in wheelchair about 12 hours a day.
7.5: Unable to take more than a few steps, restricted to wheelchair, may need aid to transfer; wheels self, but may require motorized chair for full day's activities.
8.0: Essentially restricted to bed, chair or wheelchair, but may be out of bed much of day; retains self care functions, generally effective use of arms.
8.5: Essentially restricted to bed much of day, some effective use of arms, retains some self care functions.
9.0: Helpless bed patient, can communicate and eat.
9.5: Unable to communicate effectively or eat/swallow.
10.0: Death due to MS.

Therefore, in one embodiment, treatment according to a method of the invention inhibits disability progression in a subject with MS or a variant thereof as measured on the EDSS or equivalent scale.

In another embodiment, progression of a neurological and/or psychological effect of the demyelination condition may be inhibited by treatment according to a method of the invention. As noted above, MS can have many neurological and/or psychological effects. Examples of such neurological and/or psychological effects, the progression of which may be inhibited, include, but are not limited to, depression, mood swings, emotional lability, euphoria, bipolar syndrome, anxiety, psychosis, cognitive impairments such as short-term and long-term memory problems, forgetfulness, slow word recall, aphasia and dysphasia (impairments to speech comprehension and production), neuropathic pain and dyskinesia.

In yet another embodiment of the invention, a method is provided for enhancing physical ability of a human subject having a demyelination condition. Enhancing physical ability refers generally to increasing a subject's capacity for movement, such as by increasing muscle strength, tone and/or energy. Examples of physical ability which may be enhanced by the present invention include, but are not limited to, a subject's ability to walk (ambulatory movement), coordination and balance, or a subject's use of an arm and/or facial muscles. In a particular embodiment, a subject's physical ability is enhanced such that the subject is more ambulatory as measured by the EDSS or equivalent scale.

The compound administered according to the present method is a compound of Formula (I) as set forth above, or a pharmaceutically acceptable salt thereof. In the description of Formula (I) and elsewhere in the present specification unless otherwise indicated, various groups or substituents can include the following.

Alkyl groups include straight-chain or branched saturated hydrocarbyl substituents typically containing 1 to about 20, more typically 1 to about 8, and even more typically 1 to about 6, carbon atoms.

Lower alkyl groups include alkyl substituents containing 1 to 6, especially 1 to 3, carbon atoms, and may be straight-chain or branched. Examples include methyl, ethyl, propyl, isopropyl, butyl, isobutyl, tertiary butyl, pentyl, hexyl and the like, and isomers thereof.

Alkenyl groups include straight-chain or branched hydrocarbyl substituents containing one or more double bonds and typically 2 to about 20, more typically 2 to about 8, and even more typically 2 to about 6, carbon atoms. Alkenyl groups, where asymmetric, can have cis or trans configuration.

Lower alkenyl groups include alkenyl substituents containing 2 to 6 carbon atoms that may be straight-chained or branched and in the Z or E form. Examples include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g., 1,3- or 2,4-pentadienyl, and the like.

Alkynyl groups include straight-chain or branched hydrocarbyl substituents containing one or more triple bonds and typically 2 to about 20, more typically 2 to about 8, and even more typically 2 to about 6, carbon atoms.

Lower alkynyl groups include alkynyl substituents containing 2 to 6 carbon atoms that may be straight-chained or branched. Examples include ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like.

Cycloalkyl groups include completely or partially saturated alicyclic hydrocarbyl groups containing 3 to about 18 ring carbon atoms. Cycloalkyl groups may be monocyclic or polycyclic. Examples include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalinyl, hydroindanyl, indanyl, fenchyl, pinenyl, adamantyl and the like. Cycloalkyl includes cis or trans forms. Cycloalkyl groups may be unsubstituted or mono- or polysubstituted with electron-withdrawing and/or electron-donating groups as described below. Substituents may be in endo- or exo-positions in bridged bicyclic systems. Lower cycloalkyl groups have 3 to 6 carbon atoms.

Alkoxy groups are —O-alkyl groups. Lower alkoxy groups include alkoxy substituents containing 1 to 6, especially 1 to 3, carbon atoms, and may be straight-chain or branched. Examples include methoxy, ethoxy, propoxy, butoxy, isobutoxy, tert-butoxy, pentoxy, hexoxy and the like.

Aryl groups include aromatic groups containing about 6 to about 18 ring carbon atoms, and include polynuclear aromatics. Aryl groups may be monocyclic or polycyclic, and are optionally fused. Polynuclear aromatic groups herein encompass bicyclic and tricyclic fused aromatic ring systems containing about 10 to about 18 ring carbon atoms. Aryl groups include phenyl, polynuclear aromatic groups (e.g., naphthyl, anthracenyl, phenanthrenyl, azulenyl and the like), and groups such as ferrocenyl. Aryl groups may be unsubstituted or mono- or polysubstituted with electron-withdrawing and/or electron-donating groups as described below.

Aryl lower alkyl groups include, for example, benzyl, phenylethyl, phenylpropyl, phenylisopropyl, phenylbutyl, diphenylmethyl, 1,1-diphenylethyl, 1,2-diphenylethyl and the like.

Halo or halogen groups include fluoro, chloro, bromo and iodo radicals. The prefix “halo” indicates that the substituent to which the prefix is attached is substituted with one or more independently selected halogen radicals. For example, “haloalkyl” means an alkyl substituent wherein at least one hydrogen radical is replaced with a halogen radical. Examples of haloalkyl substituents include chloromethyl, 1-bromoethyl, fluoromethyl, difluoromethyl, trifluoromethyl, 1,1,1-trifluoroethyl and the like. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), 1,1,1-trifluoroethoxy and the like. It should be recognized that if a substituent is substituted with more than one halogen radical, those halogen radicals may be identical or different, unless otherwise stated.

Carbalkoxy groups include —CO—O-alkyl groups, wherein alkyl may be lower alkyl as described above.

Acyl groups include alkanoyl groups containing 1 to about 20, more typically 1 to about 6 carbon atoms, and may be straight-chain or branched. Acyl groups include, for example, formyl, acetyl, propionyl, butyryl, isobutyryl, tertiary butyryl, pentanoyl and isomers thereof, and hexanoyl and isomers thereof.

The terms “electron-withdrawing” and “electron-donating” are well understood by one skilled in the art and are discussed, for example, in March (1985), Advanced Organic Chemistry, New York: John Wiley & Sons, at pp. 16-18, the disclosure of which is incorporated herein by reference. Electron-withdrawing groups include halo (including fluoro, chloro, bromo, and iodo), nitro, carboxy, lower alkenyl, lower alkynyl, formyl, carboxyamido, aryl, quaternary ammonium, haloalkyl (such as trifluoromethyl), aryl lower alkanoyl, carbalkoxy and the like. Electron-donating groups include hydroxy, lower alkoxy (including methoxy, ethoxy and the like), lower alkyl (including methyl, ethyl, and the like), amino, lower alkylamino, di(lower alkyl)amino, aryloxy (such as phenoxy), mercapto, lower alkylthio, lower alkylmercapto, disulfide (lower alkyldithio) and the like. One of ordinary skill in the art will appreciate that some of the aforesaid substituents may be considered to be electron-donating or electron-withdrawing under different chemical conditions. Moreover, the present invention contemplates any combination of substituents selected from the above-identified groups.

The term “heterocyclic” means a ring substituent that contains one or more sulfur, nitrogen and/or oxygen ring atoms. Heterocyclic groups include heteroaromatic groups and saturated and partially saturated heterocyclic groups. Heterocyclic groups may be monocyclic, bicyclic, tricyclic or polycyclic and can be fused rings. They typically contain up to 18 ring atoms, including up to 17 ring carbon atoms, and can contain in total up to about 25 carbon atoms, but most typically are 5- to 6-membered rings. Heterocyclic groups also include the so-called benzoheterocyclics. Representative heterocyclic groups include furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imadazolindinyl, pyrrolidinyl, furazanyl, N-methylindolyl, methylfuryl, pyridazinyl, pyrimidinyl, pyrazinyl, pyridyl, epoxy, aziridino, oxetanyl and azetidinyl groups, as well as N-oxides of nitrogen-containing heterocyclics, such as the N-oxides of pyridyl, pyrazinyl and pyrimidinyl groups and the like. Heterocyclic groups may be unsubstituted or mono- or polysubstituted with electron-withdrawing and/or electron-donating groups.

In one embodiment, a heterocyclic group is selected from thienyl, furyl, pyrrolyl, benzofuryl, benzothienyl, indolyl, methylpyrrolyl, morpholinyl, pyridyl, pyrazinyl, imidazolyl, pyrimidinyl, and pyridazinyl, especially furyl, pyridyl, pyrazinyl, imidazolyl, pyrimidinyl and pyridazinyl, more especially from furyl and pyridyl.

In another embodiment, a heterocyclic group is selected from furyl, optionally substituted with at least one lower alkyl group (preferably one having 1-3 carbon atoms, for example methyl), pyrrolyl, imidazolyl, pyridyl, pyrazinyl, pyrimidinyl, oxazolyl and thiazolyl, especially furyl, pyridyl, pyrazinyl, pyrimidinyl, oxazolyl and thiazolyl, more especially furyl, pyridyl, pyrimidinyl and oxazolyl.

Illustratively, in the compound of Formula (I), n is 1, but di- (n=2), tri- (n=3) and tetrapeptides (n=4) are also contemplated to be useful herein.

R in the compound of Formula (I) is illustratively aryl lower alkyl, especially benzyl where the phenyl ring thereof is unsubstituted or substituted with one or more electron-donating groups and/or electron-withdrawing groups, such as halo (e.g., fluoro).

R₁ in the compound of Formula (I) is preferably hydrogen or lower alkyl, especially methyl.

Particularly suitable electron-withdrawing and/or electron-donating substituents are halo, nitro, alkanoyl, formyl, arylalkanoyl, aryloyl, carboxyl, carbalkoxy, carboxamido, cyano, sulfonyl, sulfoxide, heterocyclic, guanidine, quaternary ammonium, lower alkenyl, lower alkynyl, sulfonium salts, hydroxy, lower alkoxy, lower alkyl, amino, lower alkylamino, di(lower alkyl)amino, amino lower alkyl, mercapto, mercaptoalkyl, alkylthio and alkyldithio. The term “sulfide” encompasses mercapto, mercapto alkyl and alkylthio, while the term “disulfide” encompasses alkylthio. Preferred electron-withdrawing and/or electron-donating groups are halo and lower alkoxy, especially fluoro and methoxy. These preferred substituents may be present in any one or more of the groups R, R₁, R₂, R₃, R₄, R₅, R₆, R′₆, R₇ or R₃ as defined herein.

Z—Y groups representative of R₂ and/or R₃ include hydroxy, alkoxy (such as methoxy and ethoxy), aryloxy (such as phenoxy), thioalkoxy (such as thiomethoxy and thioethoxy), thioaryloxy (such as thiophenoxy), amino, alkylamino (such as methylamino and ethylamino), arylamino (such as anilino), lower dialkylamino (such as dimethylamino), trialkylammonium salt, hydrazino, alkylhydrazino and arylhydrazino (such as N-methylhydrazino and N-phenylhydrazino), carbalkoxy hydrazino, aralkoxycarbonyl hydrazino, aryloxycarbonyl hydrazino, hydroxylamino (such as N-hydroxylamino (—NHOH), lower alkoxyamino (NHOR₁₈ wherein R₁₈ is lower alkyl, e.g., methyl), N-lower alkylhydroxylamino (N(R₁₈)OH wherein R₁₈ is lower alkyl), N-lower alkyl-O-lower alkylhydroxylamino (N(R₁₈)OR₁₉ wherein R₁₈ and R₁₉ are independently lower alkyl) and O-hydroxylamino (—O—NH₂)), alkylamido (such as acetamido), trifluoroacetamido and heterocyclylamino (such as pyrazoylamino).

Preferred heterocyclic groups representative of R₂ and/or R₃ are monocyclic 5- or 6-membered heterocyclic moieties of the formula

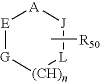

including unsaturated, partially and fully saturated forms thereof, wherein n is 0 or 1; R₅₀ is hydrogen or an electron-withdrawing or electron-donating group; A, E, L, J and G are independently CH, or a heteroatom selected from the group consisting of N, O and S; but when n is 0, G is CH, or a heteroatom selected from the group consisting of N, O and S; with the proviso that at most two of A, E, L, J and G are heteroatoms.

If n is 0, the above monocyclic heterocyclic ring is 5-membered, while if n is 1, the ring is 6-membered.

If the ring depicted hereinabove contains a nitrogen ring atom, then the N-oxide forms are also contemplated to be within the scope of the invention.

When R₂ or R₃ comprises a heterocyclic group of the above formula, it may be bonded to the main chain by a ring carbon atom. When n is 0, R₂ or R₃ may additionally be bonded to the main chain by a nitrogen ring atom.

Other preferred moieties of R₂ and R₃ are hydrogen, aryl (e.g., phenyl), arylalkyl (e.g., benzyl), and alkyl. Such moieties can be unsubstituted or mono- or polysubstituted with electron-withdrawing and/or electron-donating groups. In various embodiments, R₂ and R₃ are independently hydrogen; lower alkyl, either unsubstituted or substituted with one or more electron-withdrawing and/or electron-donating groups such as lower alkoxy (e.g., methoxy, ethoxy, and the like); N-hydroxylamino; N-lower alkylhydroxyamino; N-lower alkyl-O-lower alkyl; or alkylhydroxylamino.

In some embodiments, one of R₂ and R₃ is hydrogen.

In one embodiment n in Formula (I) is 1 and one of R₂ and R₃ is hydrogen. Illustratively in this embodiment, R₂ is hydrogen and R₃ is lower alkyl or Z—Y where Z is O, NR₄ or PR₄, and Y is hydrogen or lower alkyl; or Z—Y is NR₄NR₅R₇, NR₄OR₅, ONR₄R₇,

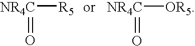

In another embodiment, n is 1, R₂ is hydrogen, and R₃ is lower alkyl which is unsubstituted or substituted with an electron-withdrawing or electron-donating group, NR₄OR₅ or ONR₄R₇.

In yet another embodiment,
n is 1;
R is aryl lower alkyl, which aryl group is unsubstituted or substituted with an electron-withdrawing group, for example aryl can be phenyl, which is unsubstituted or substituted with halo;
R₁ is lower alkyl;
R₂ is hydrogen; and
R₃ is lower alkyl which is unsubstituted or substituted with hydroxy, lower alkoxy, NR₄OR₅ or ONR₄R₇, wherein R₄, R₅ and R₇ are independently hydrogen or lower alkyl.

In yet another embodiment, R₂ is hydrogen and R₃ is hydrogen, an alkyl group which is unsubstituted or substituted with at least one electron-withdrawing or electron-donating group or Z—Y. In this embodiment, R₃ is illustratively hydrogen, an alkyl group such as methyl, which is unsubstituted or substituted with an electron-donating group such as lower alkoxy, more especially methoxy or ethoxy, or with NR₄OR₅ or ONR₄R₇, wherein R₄, R₅ and R₇ are independently hydrogen or lower alkyl.

In yet another embodiment, R₂ and R₃ are independently hydrogen, lower alkyl, or Z—Y; Z is O, NR₄ or PR₄; Y is hydrogen or lower alkyl; or Z—Y is NR₄NR₅R₇, NR₄OR₅, ONR₄R₇,

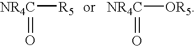

It is preferred that R is aryl lower alkyl. The most preferred aryl for R is phenyl. The most preferred R group is benzyl. The aryl group is unsubstituted or substituted with an electron-withdrawing or electron-donating group. If the aryl ring in R is substituted, it is most preferred that it is substituted with an electron-withdrawing group. The most preferred electron-withdrawing group for R is halo, especially fluoro.

The preferred R₁ is lower alkyl, especially methyl.

In one embodiment R is aryl lower alkyl, e.g., benzyl, and R₁ is lower alkyl, e.g., methyl.

Further preferred compounds are compounds of Formula (I) wherein
n is 1;
R is aryl or aryl lower alkyl, such as benzyl, wherein the aryl group is unsubstituted or substituted with an electron-withdrawing or electron-donating group;
R₁ is lower alkyl;
R₂ is hydrogen; and
R₃ is hydrogen, a lower alkyl group, especially methyl which is substituted with an electron-withdrawing or electron-donating group, or Z—Y.
   In this embodiment, it is more preferred that R₃ is hydrogen, a lower alkyl group, especially methyl, which may be substituted with an electron-donating group such as lower alkoxy (e.g., methoxy, ethoxy or the like), NR₄OR₅ or ONR₄R₇ wherein these groups are as defined hereinabove.

In one aspect, the compound is represented by Formula (II)

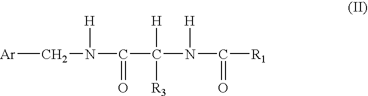

or a pharmaceutically acceptable salt thereof, wherein
Ar is aryl, especially phenyl, which is unsubstituted or substituted with at least one halo;
R₁ is lower alkyl, especially C₁₋₃ alkyl, for example methyl; and
R₃ is hydrogen or lower alkyl, which is unsubstituted or substituted with at least one electron-withdrawing or electron-donating group or Z—Y; for example R₃ is —CH₂-Q, wherein Q is lower alkoxy, especially C₁₋₃ alkoxy, for example methoxy.

In another aspect, the compound has Formula (I) wherein
n is 1;
R is unsubstituted or substituted benzyl, in particular halo-substituted benzyl;
R₁ is lower alkyl, especially C₁₋₃ alkyl, for example methyl;
R₂ is hydrogen; and
R₃ is as broadly defined herein.

In yet another aspect, the compound is represented by Formula (III)

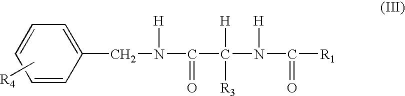

or a pharmaceutically acceptable salt thereof, wherein
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide;
R₃ is selected from the group consisting of hydrogen, alkyl, alkoxy, alkoxyalkyl, aryl, N-alkoxy-N-alkylamino, and N-alkoxyamino; and
R₁ is alkyl.

Alkyl, alkoxy, alkenyl and alkynyl groups in a compound of Formula (III) are lower alkyl, alkoxy, alkenyl and allynyl groups having no more than 6, more typically no more than 3, carbon atoms.

In a particular aspect, R₄ substituents in a compound of Formula (III) are independently selected from hydrogen and halo, more particularly fluoro, substituents.

In a particular aspect, R₃ in a compound of Formula (III) is alkoxyalkyl, phenyl, N-alkoxy-N-alkylamino or N-alkoxyamino.

In a particular aspect, R₁ in a compound of Formula (III) is C₁₋₃ alkyl.

In a more particular aspect, no more than one R₄ substituent is fluoro and all others are hydrogen; R₃ is selected from the group consisting of methoxymethyl, phenyl, N-methoxy-N-methylamino and N-methoxyamino; and R₁ is methyl.

It is to be understood that combinations and permutations of R₁, R₂, R₃ and R groups and values of n, even if such combinations and permutations are not explicitly described herein, are contemplated to be within the scope of the present invention. Moreover, the present invention also encompasses methods that comprise administering a compound having one or more elements of each of the Markush groupings described for R₁, R₂, R₃ and R and the various combinations thereof. Thus, for example, the present invention contemplates that R₁ and R may independently be one or more of the substituents listed hereinabove in combination with any of the R₂ and R₃ substituents, independently with respect to each of the n

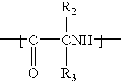

subunits of the compound of Formula (I).

Compounds useful herein may contain one or more asymmetric carbons and may exist in optically active forms. The configuration around each asymmetric carbon can be either the D or L configuration. Configuration around a chiral carbon atom can also be described as R or S in the Cahn-Prelog-Ingold system. All of the various configurations around each asymmetric carbon, including the various enantiomers and diastereomers as well as mixtures of enantiomers, diastereomers or both, including but not limited to racemic mixtures, are contemplated by the present invention.

More particularly, in a compound of Formula (I) where R₂ and R₃ are not identical, there exists asymmetry at the carbon atom to which the groups R₂ and R₃ are attached. As used herein, the term “configuration” generally refers to the configuration around the carbon atom to which R₂ and R₃ are attached, even though other chiral centers may be present in the molecule. Therefore, unless the context demands otherwise, when referring to a particular configuration such as D or L, it is to be understood to mean the D- or L-stereoisomer at the carbon atom to which R₂ and R₃ are attached. However, all possible enantiomers and diastereomers at other chiral centers, if any, present in the compound are encompassed herein.

The compounds useful herein can comprise the L- or D-stereoisomer as defined above, or any mixture thereof, including without limitation a racemic mixture. The D-stereoisomer is generally preferred. In lacosamide, the D-stereoisomer corresponds to the R-enantiomer according to R,S terminology.

In one embodiment the compound, for example lacosamide, is substantially enantiopure. As used herein, the term “substantially enantiopure” means having at least 88%, for example at least 90%, more preferably at least 95%, 96%, 97%, 98% or 99%, enantiomeric purity.

Illustrative compounds that can be used according to the present method include:
(R)-2-acetamido-N-benzyl-3-methoxypropionamide (lacosamide);
(R)-2-acetamido-N-benzyl-3-ethoxypropionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamide acetic acid benzylamide; and
D-1,2-(O-methylhydroxylamino)-2-acetamide acetic acid benzylamide.

Depending upon the substituents, certain of the present compounds may form salts. For example, some compounds of Formulas (I), (II) and (III) can form salts with a wide variety of acids, inorganic and organic, including pharmaceutically acceptable acids. Such salts can have enhanced water solubility and may be particularly useful in preparing pharmaceutical compositions for use in situations where enhanced water solubility is advantageous.

Pharmaceutically acceptable salts are those having therapeutic efficacy without unacceptable toxicity. Salts of inorganic acids such as hydrochloric, hydroiodic, hydrobromic, phosphoric, metaphosphoric, perchloric, nitric and sulfuric acids as well as salts of organic acids such as tartaric, acetic, citric, malic, benzoic, glycolic, gluconic, succinic, arylsulfonic (e.g., p-toluene sulfonic, benzenesulfonic) and malonic acids and the like, can be used.

Compounds useful herein can be prepared by any known procedure of synthesis.

Above-cited U.S. Pat. No. 5,378,729 describes procedures for synthesis of compounds of Formula (I).

Above-cited U.S. Pat. No. 5,773,475 describes procedures for synthesis of compounds of Formula (I).

Without being bound by theory, it is now thought that (R)-2-acetamido-N-benzyl-3-methoxypropionamide (lacosamide) may act at least in part by modulation of collapsin response mediator protein 2 (CRMP-2). A poster by Stoehr et al. presented at the 9th International Conference on the Mechanisms and Treatment of Neuropathic Pain, Bermuda, November 2006, not admitted to be prior art to the present invention, states that “[l]acosamide modulates CRMP-2 and as a consequence attenuates the effects of neurotrophic factors on axon growth.” It is further stated that “[t]he interaction of lacosamide with CRMP-2 represents a second mode of action of lacosamide and this might potentially result in disease modifying effects.”

In some embodiments, the method of the present invention further comprises administering to the subject at least one further active agent for treatment of multiple sclerosis or a variant thereof.

In another embodiment of the invention, a therapeutic combination is provided comprising
(a) a compound of Formula (I), (II) or (III), for example lacosamide, or a pharmaceutically acceptable salt thereof, and
(b) at least one further active agent for treatment of multiple sclerosis or a variant thereof.

The term “therapeutic combination” refers to a plurality of agents that, when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

The compound of Formula (I), (II) or (III), for example lacosamide, and the at least one further active agent for treatment of multiple sclerosis or a variant thereof can be administered together, i.e., in a single co-formulated dosage form, or separately, i.e., as components of two separate dosage forms. Separate dosage forms can be administered substantially at the same time or at different times or frequencies.

Further, the two or more active agents of a therapeutic combination can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, the compound of Formula (I), (II) or (III) and, in a second container, the at least one further active agent for treatment of multiple sclerosis or a variant thereof. In another example, the compound of Formula (I), (II) or (III) and the at least one further active agent for treatment of multiple sclerosis or a variant thereof are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the compound of Formula (I), (II) or (III) and the at least one further active agent for treatment of multiple sclerosis or a variant thereof may be administered on the same or on different schedules, for example on a daily, weekly or monthly basis.

In a further embodiment of the invention, a pharmaceutical composition is provided comprising
(a) a compound of Formula (I), (II) or (III), for example lacosamide, or a pharmaceutically acceptable salt thereof, and
(b) at least one further active agent for treatment of multiple sclerosis or a variant thereof.
   The pharmaceutical composition can include any pharmaceutically acceptable excipient, for example selected from those provided hereinbelow.

Examples of the at least one further active agent for treatment of multiple sclerosis or a variant thereof include, but are not limited to, interferon β, glatiramer acetate, mitoxantrone, teriflunomide, testosterone, fingolimod, temsirolimus, BHT-3009, MBP-8298, IR-208, CDP-323, cladribine, laquinimod, monoclonal antibodies, statins such as atorvastatin, cerivastatin, fluvastatin, lovastatin, pitavastatin, pravastatin, rosuvastatin and simvastatin, and corticosteroids. Other agents, including specific antibodies, are in development for treatment of multiple sclerosis.

Suitable regimens including doses and routes of administration for the at least one further active agent for treatment of multiple sclerosis or a variant thereof can be determined from readily-available reference sources relating to these agents, for example Physicians' Desk Reference (PDR), 60th edition, Montvale, N.J.: Thomson (2006), and various internet sources known to those of skill in the art. When administered in combination with a compound of Formula (I), (II) or (III), for example lacosamide, the at least one further active agent for treatment of multiple sclerosis or a variant thereof can be used at a full dose, but the physician may elect to administer less than a full dose of the at least one further active agent, at least initially.

A compound of Formula (I), (II) or (III) as described herein, is used at a dose and frequency effective to inhibit demyelination and/or at a therapeutically effective dose. A physician can determine a suitable dosage of a compound, which can vary with the particular compound chosen, the route and method of administration, and the age and other characteristics of the individual patient. The physician can initiate treatment with small doses, for example substantially less than an optimum dose of the compound, and increase the dose by small increments until an optimum effect under the circumstances is achieved. When the composition is administered orally, larger quantities of the compound may be required to produce the same therapeutic benefit as a smaller quantity given parenterally.

In a particular aspect, the compound, for example lacosamide, is administered in an amount ranging from about 1 mg to about 10 mg per kilogram of body weight per day. Typically a patient can be treated with the compound, for example lacosamide, at a dose of at least about 50 mg/day, for example at least about 100 mg/day, at least about 200 mg/day, at least about 300 mg/day or at least about 400 mg/day. Generally, a suitable dose is not greater than about 6 g/day, for example not greater than about 1 g/day or not greater than about 600 mg/day. In some cases, however, higher or lower doses may be needed.

In another aspect, the daily dose is increased until a maintenance dose is reached which is maintained during further treatment. A maintenance dose refers to a dose that provides a useful effect to a subject with a demyelination condition and is tolerated by the subject. A maintenance dose may vary by subject.

In yet another aspect, several divided doses are administered daily. For example, no more than three doses per day, or no more than two doses per day, may be administered. However, it is often most convenient to administer no more than a single dose per day.

Doses expressed herein on a daily basis, for example in mg/day, are not to be interpreted as requiring a once-a-day frequency of administration. For example, a dose of 300 mg/day can be given as 100 mg three times a day, or as 600 mg every second day.

In yet another aspect, an amount of the compound, for example lacosamide, is administered which results in a plasma concentration of the compound of about 0.1 to about 15 μg/ml (steady-state trough) and about 5 to about 18.5 μg/ml (steady-state peak). This may be calculated as an average over a plurality of treated subjects.

Many demyelination conditions, such as multiple sclerosis, are difficult to clinically diagnose in early stages of the condition. For example, multiple sclerosis requires at least two demyelinating events to occur at least about 30 days apart before a definitive clinical diagnosis can be made. The interval between demyelinating events may be longer than 30 days. However, the earlier that treatment begins, the better. It is therefore contemplated that, in some embodiments, administration of a compound of Formula (I), (II) or (III), for example lacosamide, is initiated before a definitive clinical diagnosis is made, for example before secondary effects such as dyskinesia, neuropathic pain or psychosis are evident, but generally after at least a first demyelinating event.

Demyelination is a chronic process. Administration of a compound of Formula (I), (II) or (III), for example lacosamide, should therefore, in some embodiments, continue for an extended period of time, typically at least about 1 month, more typically at least about 3 months. Duration of therapy depends on the type of demyelination condition, for example the type of multiple sclerosis, and in some embodiments can be at least about 1 year, at least about 5 years, or for as long as needed, which can be lifelong (i.e., from a time of initiation of treatment for substantially the remainder of the patient's life). In a particular embodiment, a compound of Formula (I), (II) or (III), for example lacosamide, is administered for at least about 3 months. Duration of therapy is an important consideration where, as in certain embodiments of the present invention, it is an objective to modify an underlying disease process such as demyelination, not merely to provide palliative treatment of symptoms or outward effects of a disease.

The compound of Formula (I), (II) or (III), for example lacosamide, can be administered in any convenient and effective manner, such as by oral, intravenous, intraperitoneal, intramuscular, intrathecal, subcutaneous or transmucosal (e.g., buccal or intranasal) routes. Oral or intravenous administration is generally preferred.

For oral administration, the compound is typically administered as a component of an orally deliverable pharmaceutical composition that further comprises an inert diluent or an assimilable edible carrier, or it may be incorporated into the subject's food or water. In an orally deliverable pharmaceutical composition, the compound can be incorporated together with one or more excipients and administered in the form of tablets, troches, pills, capsules, elixirs, suspensions, syrups, wafers or the like. Such compositions typically contain at least about 1%, more typically about 5% to about 80%, by weight of the compound, for example lacosamide. The amount of the compound in the composition is such that, upon administration of the composition, a suitable dosage as set forth above can conveniently be provided. Illustratively, a pharmaceutical composition useful for oral delivery of a compound of Formula (I), (II) or (III), for example lacosamide, contains per dose about 10 mg to about 6 g, for example about 50 to about 1000 mg, or about 100 to about 600 mg, of the compound.

In particular embodiments the composition is enclosed in hard- or soft-shell (e.g., gelatin) capsules, or is in a form of compressed or molded tablets. The composition illustratively comprises as excipients one or more of a diluent such as lactose or dicalcium phosphate (in the case of capsules a liquid carrier can be present); a binding agent such as gum tragacanth, acacia, corn starch or gelatin; a disintegrating agent such as corn starch, potato starch, alginic acid or the like; and a lubricant such as magnesium stearate. A sweetening agent such as sucrose or saccharin and/or a flavoring agent such as peppermint, oil of wintergreen, or cherry flavoring can be added if desired.

Various other excipients may be present as coatings or otherwise modifying the physical form of the composition. For example, tablets, pills or capsules may be coated with shellac, sugar or both. A syrup or elixir may contain the active compound, sucrose as a sweetening agent, methyl- and propylparabens as preservatives, a dye, and flavoring such as cherry or orange flavor. The active compound can be incorporated into a sustained-release formulation. For example, sustained-release dosage forms are contemplated wherein the compound is bound to an ion exchange resin which, optionally, can be coated with a diffusion barrier coating to modify the release properties of the resin.

Pharmaceutical compositions suitable for injection include sterile aqueous solutions (where the compound is water-soluble), dispersions, and sterile powders for extemporaneous preparation of sterile injectable solutions or dispersions. In such cases the injectable composition must be sterile and must be sufficiently fluid to permit easy syringeability. The composition must be stable under the conditions of manufacture and storage and must typically be preserved against the contaminating action of microorganisms such as bacteria and fungi. The carrier can be a solvent or dispersion medium containing, for example, water, ethanol, a polyol (for example, glycerol, propylene glycol, liquid polyethylene glycol, or the like), suitable mixtures thereof, or a vegetable oil. Microbial action can be inhibited by various antibacterial and antifungal agents, for example parabens, chlorobutanol, phenol, sorbic acid, thimerosal or the like. In many cases, it will be preferable to include tonicity agents, for example, sugars or sodium chloride, to provide a substantially isotonic liquid for injection. Prolonged absorption of injectable compositions can be brought about by use in the compositions of agents delaying absorption, for example aluminum monostearate or gelatin.

Sterile injectable solutions can be prepared by incorporating the active compound in a required amount in an appropriate solvent with various other ingredients mentioned above, as required, followed by filtered sterilization. Generally, dispersions are prepared by incorporating sterilized active compound into a sterile vehicle which contains the dispersion medium and other excipient ingredients such as those mentioned above. Sterile powders for preparation of sterile injectable solutions can be prepared by vacuum-drying or freeze-drying a previously sterile-filtered solution or dispersion.

A further subject of the present invention is the use of a compound described herein for the production of a pharmaceutical composition for inhibiting demyelination in a demyelination condition as described herein. The compound described herein can be used for the production of a pharmaceutical composition for delaying clinical onset of a demyelination condition in a human subject. The compound described herein can also be used for the production of a pharmaceutical composition for inhibiting progression and/or reducing frequency of relapse of a demyelination condition in a human subject. The compound described herein can also be used for the production of a pharmaceutical composition for enhancing physical ability of a human subject having a demyelination condition. A particular embodiment is the use of a compound as described herein for the production of a pharmaceutical composition for the treatment of multiple sclerosis or a variant thereof, as described herein. A further particular embodiment is the use of a compound as described herein for the production of a pharmaceutical composition for inhibiting demyelination in multiple sclerosis or a variant thereof, as described herein.

A further subject of the present invention is a pharmaceutical composition comprising a compound as described herein for inhibiting demyelination in a demyelination condition as described herein. The pharmaceutical composition comprising a compound as described herein may be suitable for delaying clinical onset of a demyelination condition in a human subject. The pharmaceutical composition comprising a compound as described herein may also be suitable for inhibiting progression and/or reducing frequency of relapse of a demyelination condition in a human subject. The pharmaceutical composition comprising a compound as described herein may also be suitable for enhancing physical ability of a human subject having a demyelination condition. A particular embodiment is a pharmaceutical composition comprising a compound as described herein for the treatment of multiple sclerosis or a variant thereof, as described herein. A further particular embodiment is a pharmaceutical composition comprising a compound as described herein for inhibiting demyelination in multiple sclerosis or a variant thereof, as described herein.

Yet another subject of the present invention is the use of a compound as described herein for inhibiting demyelination in a demyelination condition as described herein. The compound as described herein may be used for delaying clinical onset of a demyelination condition in a human subject. The compound as described herein may also be used for inhibiting progression and/or reducing frequency of relapse of a demyelination condition in a human subject. The compound as described herein may also be used for enhancing physical ability of a human subject having a demyelination condition. A particular embodiment is the use of a compound as described herein for the treatment of multiple sclerosis or a variant thereof, as described herein. A further particular embodiment is the use of a compound as described herein for inhibiting demyelination in multiple sclerosis or a variant thereof, as described herein.

### Example

### Acute Experimental Allergic Encephalomyelitis (“EAE”) Rat Model

The utility of a compound of Formula (I), (II) or (III), for example lacosamide, for inhibiting demyelination in multiple sclerosis is assessed in a study using the acute EAE model. EAE is an autoimmune CNS demyelination condition that mimics many of the clinical and pathologic features of multiple sclerosis. The EAE rat model is well known in the art and has been used as a model of multiple sclerosis since its development in the 1930s. See, for example, the publications individually cited below.
Van Epps (2005) J. Exp. Med. 202(1):4.
Kabat et al. (1946) J. Exp. Med. 85:117-130.

EAE is induced in female Lewis rats on day zero of the study by a single inoculum injection of myelin basic protein (MBP) and complete Freund's adjuvant (CFA) containing heat killed Mycobacterium tuberculosis F137 Ra at a concentration of 4 mg/ml (MD Biosciences Ltd, Israel). This MBP/CFA encephalitogenic emulsive inoculum (100 μg MBP+200 μg CFA) is injected at a total dose volume of 100 μl/animal, delivered as 2×50 subcutaneous bilateral injections into the intraplanar paw regions.

Lacosamide is administered by intraperitoneal (i.p.) injection twice daily (b.i.d.) on days 0-21 of the study, in a volume of 10 ml/kg at daily doses of 6, 20 and 60 mg/kg. A vehicle control containing no lacosamide is administered by the same route and at the same frequency. An additional group of animals receives i p administration of the positive reference compound dexamethasone once daily at 0.5 or 1 mg/kg.

The duration of the study is 21 days. Careful clinical examinations are carried out and recorded at least once daily in addition to EAE clinical scoring and assessment. Observations made include changes in skin, fur, eyes and mucous membranes, occurrence of secretions and excretions (e.g., diarrhea) and autonomic activity (e.g., lacrimation, salivation, piloerection, pupil size and unusual respiratory pattern), gait, posture and response to handling, as well as presence of bizarre behavior, tremors, convulsions, sleep and coma.

Body weight loss can be the first sign of disease initiation, while a sudden marked weight gain tends to accompany remission of EAE symptoms. Therefore, determination of individual body weights of animals is made shortly before EAE induction on day 0 (study commencement) and thereafter on a daily basis throughout the entire 21-day observation period.

Initially, all animals are examined for signs of any neurological responses and symptoms prior to EAE induction (day 0) and thereafter examined on a daily basis through the entire 21-day observation period. To avoid experimental bias, EAE reactions are determined in a blinded fashion, as much as possible, by a staff member unaware of the specific treatment applied. EAE reactions are scored and recorded on a classical 0-5 scale in ascending order of severity as shown below:
0: no abnormalities;
0.5: tail weakness—distal half;
1: tail weakness—proximal half;
1.5: hind paw weakness—one paw;
2: hind paw weakness—two paws;
2.5: fore paw paralysis—one paw;
3: fore paw paralysis—two paws;
4: full paralysis;
5: death.

Evaluation is primarily based on relative recorded changes in both neurological symptoms and body weights, expressed as absolute values, percentage change and mean group values obtained in all treated groups by comparison with those of the vehicle control.

In a study conducted as described above, disease onset in vehicle-treated animals occurred at day 9 following induction of EAE. Disease onset was significantly postponed (to day 12 after EAE induction) by lacosamide at doses of 10 and 30 mg/kg b.i.d. and by the positive reference compound dexamethasone (0.5 mg/kg).

All patents and publications cited herein are incorporated by reference into this application in their entirety.

The words “comprise”, “comprises”, and “comprising” are to be interpreted inclusively rather than exclusively.

What is claimed is:

1. A method for inhibiting demyelination in a human subject having a demyelination condition, the method comprising administering to the subject a compound of Formula (I) 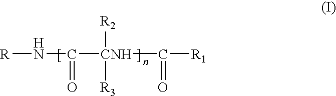
wherein
R is hydrogen, lower alkyl, lower alkenyl, lower alkynyl, aryl, aryl lower alkyl, heterocyclic, heterocyclic lower alkyl, lower alkyl heterocyclic, lower cycloalkyl or lower cycloalkyl lower alkyl, and R is unsubstituted or is substituted with at least one electron withdrawing group and/or at least one electron donating group;
R₁ is hydrogen or lower alkyl, lower alkenyl, lower alkynyl, aryl lower alkyl, aryl, heterocyclic lower alkyl, lower alkyl heterocyclic, heterocyclic, lower cycloalkyl, lower cycloalkyl lower alkyl, each unsubstituted or substituted with at least one electron donating group and/or at least one electron withdrawing group;
R₂ and R₃ are independently hydrogen, lower alkyl, lower alkenyl, lower alkynyl, aryl lower alkyl, aryl, halo, heterocyclic, heterocyclic lower alkyl, lower alkyl heterocyclic, lower cycloalkyl, lower cycloalkyl lower alkyl, or Z—Y, wherein R₂ and R₃ may be unsubstituted or substituted with at least one electron withdrawing group and/or at least one electron donating group; and wherein heterocyclic in R₂ and R₃ is furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imidazolindinyl, pyrrolidinyl, furazanyl, N-methylindolyl, methylfuryl, pyridazinyl, pyrimidinyl, pyrazinyl, pyridyl, epoxy, aziridino, oxetanyl, azetidinyl or, when N is present in the heterocyclic, an N-oxide thereof;
Z is O, S, S(O)ₐ, NR₄, NR₆′, PR₄ or a chemical bond;
Y is hydrogen, lower alkyl, aryl, aryl lower alkyl, lower alkenyl, lower alkynyl, halo, heterocyclic, heterocyclic lower alkyl, lower alkyl heterocyclic and Y may be unsubstituted or substituted with at least one electron donating group and/or at least one electron withdrawing group, wherein heterocyclic has the same meaning as in R₂ or R₃ and, provided that when Y is halo, Z is a chemical bond, or
Z—Y taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR₄NR₅R₇, N⁺R₅R₆R₇, 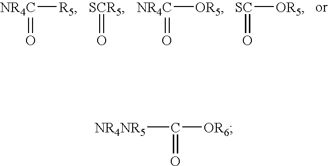
R₆′ is hydrogen, lower allyl, lower alkenyl, or lower alkynyl which may be unsubstituted or substituted with at least one electron withdrawing group and/or at least one electron donating group;
R₄, R₅ and R₆ are independently hydrogen, lower alkyl, aryl, aryl lower alkyl, lower alkenyl, or lower alkynyl, wherein R₄, R₅ and R₆ may independently be unsubstituted or substituted with at least one electron withdrawing group and/or at least one electron donating group;
R₇ is R₆ or COOR₈ or COR₈, which R₇ may be unsubstituted or substituted with at least one electron withdrawing group and/or at least one electron donating group;
R₈ is hydrogen or lower alkyl, or aryl lower alkyl, and the aryl or alkyl group may be unsubstituted or substituted with at least one electron withdrawing group and/or at least one electron donating group;
n is 1-4; and
a is 1-3,
or a pharmaceutically acceptable salt thereof; at a dose and frequency effective to inhibit demyelination when continued for a period of at least about 3 months.

2. The method of claim 1, further comprising diagnosing said demyelination condition in the subject prior to initiating administration of the compound or salt thereof.

3. The method of claim 1, wherein administration of the compound or salt thereof is initiated prior to diagnosis of said demyelination condition but after occurrence of at least one demyelinating event.

4. The method of claim 3, wherein the demyelinating event comprises one or more events independently selected from the group consisting of demyelination lesions, optic neuritis, numbness and tingling in a limb, difficulty with speech, loss of balance and coordination, and motor and sensory problems.

5. The method of claim 1, wherein the demyelination is associated with an autoimmune response.

6. The method of claim 1, wherein the demyelination condition is selected from the group consisting of multiple sclerosis and variants thereof, transverse myelitis, Guillain-Barré syndrome and progressive multifocal leukoencephalopathy.

7. The method of claim 1, wherein the demyelination condition comprises multiple sclerosis or a variant thereof selected from the group consisting of optic-spinal multiple sclerosis, neuromyelitis optica, acute disseminated encephalomyelitis, Balo concentric sclerosis, Schilder disease and Marburg multiple sclerosis.

8. The method of claim 7, wherein clinical onset of multiple sclerosis is delayed.

9. The method of claim 7, wherein the multiple sclerosis or variant thereof is relapse-remitting.

10. The method of claim 7, wherein frequency of relapse is reduced.

11. The method of claim 7, wherein the multiple sclerosis or variant thereof is primary progressive, secondary progressive or progressive relapsing.

12. The method of claim 11, wherein progression of the demyelination condition is inhibited.

13. The method of claim 12, wherein said progression comprises disability progression.

14. The method of claim 12, wherein said progression comprises progression of a neurological and/or psychological effect.

15. The method of claim 1, wherein the compound of Formula (I) is a compound of Formula (III): 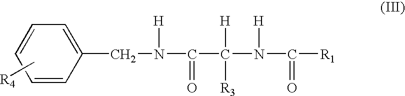
wherein:
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto and disulfide;
R₃ is selected from the group consisting of hydrogen, alkyl, alkoxy, alkoxyalkyl, aryl, N-alkoxy-N-alkylamino and N-alkoxyamino; and
R₁ is alkyl;
or a pharmaceutically acceptable salt thereof.

16. The method of claim 15, wherein, in the compound of Formula (III) or salt thereof:
R₄ is one or more substituents independently selected from the group consisting of hydrogen and halo;
R₃ is selected from the group consisting of lower alkoxy-lower alkyl, aryl, N-lower alkoxy-N-lower alkylamino, and N-lower alkoxyamino; and
R₁ is lower alkyl.

17. The method of claim 15, wherein, in the compound of Formula (III) or salt thereof, R₃ is lower alkoxy-lower alkyl.

18. The method of claim 15, wherein, in the compound of Formula (III) or salt thereof:
R₄ is hydrogen;
R₃ is methoxymethyl; and
R₁ is methyl.

19. The method of claim 15, wherein the compound of Formula (III) is selected from the group consisting of:
(R)-2-acetamido-N-benzyl-3-methoxy-propionamide (lacosamide);
(R)-2-acetamido-N-benzyl-3-ethoxy-propionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamide acetic acid benzylamide; and
D-1,2-(O-methylhydroxylamino)-2-acetamide acetic acid benzylamide.

20. The method of claim 15, wherein the compound of Formula (III) is substantially enantiopure.

21. The method of claim 15, wherein the compound of Formula (III) is lacosamide or a pharmaceutically acceptable salt thereof.

22. The method of claim 21, wherein the lacosamide is administered in a dose of about 100 to about 6000 mg/day.

23. The method of claim 21, wherein the lacosamide is administered in a dose of about 200 to about 1000 mg/day.

24. The method of claim 21, wherein the lacosamide is administered in a dose of about 300 to about 600 mg/day.

25. The method of claim 21, wherein the lacosamide is administered in increasing daily doses until a maintenance dose is reached which is maintained during further treatment.

26. The method of claim 21, wherein the lacosamide is administered in not more than three doses per day.

27. The method of claim 21, wherein the lacosamide is administered not more than once daily.

28. The method of claim 21, wherein the lacosamide is administered for a period of at least about 1 year.

29. The method of claim 21, wherein the lacosamide is administered in a pharmaceutical composition resulting in a plasma concentration of the compound of 0.1 to 15 μg/ml (steady-state trough) and 5 to 18.5 μg/ml (steady-state peak).

30. The method of claim 21, wherein the lacosamide is administered orally.

31. The method of claim 1, wherein the inhibition of demyelination is mediated at least in part by modulation of CRIMP-2.

32. The method of claim 7, further comprising administering to the subject at least one further active agent for treatment of multiple sclerosis or a variant thereof.

33. The method of claim 32, wherein the at least one further active agent comprises one or more drugs independently selected from the group consisting of interferon β, glatiramer acetate, mitoxantrone, teriflunomide, testosterone, fingolimod, temsirolimus, BHT-3009, MBP-8298, IR-208, cladribine, laquinimod, monoclonal antibodies, statins, corticosteroids and combinations thereof.

34. The method of claim 32, wherein the compound of Formula (I) or salt thereof and the at least one further active agent are administered in a single pharmaceutical composition.

35. A method for delaying clinical onset of a demyelination condition in a human subject, comprising administering to the subject a compound of Formula (I) or a pharmaceutically acceptable salt thereof in a therapeutically effective amount.

36. A method for inhibiting progression and/or reducing frequency of relapse of a demyelination condition in a human subject, comprising administering to the subject a compound of Formula (I) or a pharmaceutically acceptable salt thereof in a therapeutically effective amount for a period of at least about 3 months.

37. A method for enhancing physical ability of a human subject having a demyelination condition, comprising administering to the subject a compound of Formula (I) or a pharmaceutically acceptable salt thereof in a therapeutically effective amount for a period of at least about 3 months.

38. A therapeutic combination comprising.
(a) a compound of Formula (I) or a pharmaceutically acceptable salt thereof, and
(b) at least one further active agent for treatment of multiple sclerosis or a variant thereof;
the combination being contained in a single pharmaceutical composition or in separate pharmaceutical compositions respectively comprising said compound or salt thereof (a) and said further active agent(s) (b).

39. The therapeutic combination of claim 38, wherein the at least one further active agent is independently selected from the group consisting of interferon Vs, glatiramer acetate, mitoxantrone, teriflunomide, fingolimod, temsirolimus, BHT-3009, MBP-8298, IR-208, cladribine, laquinimod, monoclonal antibodies, statins and combinations thereof.

40. The therapeutic combination of claim 38, wherein (a) and (b) are contained in a single pharmaceutical composition further comprising one or more pharmaceutically acceptable excipients.

41. The method of claim 1, wherein the demyelination condition is selected from the group consisting of multiple sclerosis and variants thereof, transverse myelitis, and progressive multifocal leukoencephalopathy.
